(19) United States
(12) Patent Application Publication
(10) Pub. No.: US 2026/0041410 A1
(43) Pub. Date: Feb. 12, 2026

(54) Detection Device, Medical Imaging Apparatus, and Medical Puncture System

(71) Applicant: Shanghai United Imaging Healthcare Co., Ltd., Shanghai (CN)

(72) Inventor: Jia Yang, Shanghai (CN)

(21) Appl. No.: 19/277,439

(22) Filed: Jul. 23, 2025

(30) Foreign Application Priority Data
Aug. 12, 2024 (CN) 202411104198.8

(51) Int. Cl. A61B 10/02; A61B 90/00

(52) U.S. Cl. CPC A61B 10/0233; A61B 90/37; A61B 2090/376; A61B 2090/378

(57) ABSTRACT

The present disclosure relates to a detection device, medical imaging apparatus, and medical puncture system. The detection device comprising: a compression unit, configured to compress an object; an X-ray detector, located on one side of the compression unit along a first direction, the X-ray device adapted for acquiring an X-ray image of the object; an ultrasonic detector, spaced apart from the X-ray detector and capable of moving relative to the X-ray detector along a second direction, the ultrasonic detector adapted for acquiring an ultrasonic image of the object.

### CROSS-REFERENCE TO RELATED APPLICATIONS

This application claims priority to Chinese patent application No. 202411104198.8, filed on Aug. 12, 2024, the content of which is hereby incorporated by reference in its entirety.

### TECHNICAL FIELD

This application relates to the technical field of medical devices, particularly to a detection device, a medical imaging apparatus, and a medical puncture system.

### BACKGROUND

Current examinations for breast diseases and early screenings for breast cancer are primarily conducted through mammographic X-ray imaging or breast ultrasound imaging. The principle of mammographic X-ray imaging is similar to other X-ray devices in terms of exposure mechanisms. It mainly utilizes the penetrative properties of X-rays and the differential responses of human tissues to X-rays during penetration, ultimately forming breast images (including 2D and 3D tomographic imaging) with varying gray levels through energy accumulation. The principle of breast ultrasound imaging involves scanning the human body with ultrasonic beams and processing reflected signals to obtain images of internal organs.

X-ray imaging as an example, while it can identify pathological details such as microcalcifications and detect potential breast lesions early, it struggles to visualize the complete structural imaging of pathological tissues. Therefore, existing detection devices suffer from limited flexibility in practical use.

### SUMMARY

An aspect of the present disclosure may provide a detection device, comprising: a compression unit, configured to compress an object; an X-ray detector, located on one side of the compression unit along a first direction, the X-ray device adapted for acquiring an X-ray image of the object; an ultrasonic detector, spaced apart from the X-ray detector and capable of moving relative to the X-ray detector along a second direction, the ultrasonic detector adapted for acquiring an ultrasonic image of the object.

In some embodiments, wherein the X-ray detector is movable relative to the compression unit along the first direction wherein ultrasonic detector and the X-ray detector are positioned along the same side of the compression assembly relative to the first direction.

In some embodiments, the detection device further comprises an ultrasonic coupling medium, wherein at least a portion of the ultrasonic coupling medium (140) is disposed between the ultrasonic detector and the object.

In some embodiments, wherein the ultrasonic coupling medium comprises a liquid coupling agent; wherein the detection device further comprises a container, wherein the liquid coupling agent is arranged within the container; and wherein the ultrasonic detector is located within the container.

In some embodiments, wherein the X-ray detector is located within the container.

In some embodiments, wherein the ultrasonic coupling medium comprises a first solid ultrasonic coupling material disposed between the ultrasonic detector and the object.

In some embodiments, wherein the ultrasonic coupling medium further comprises a second solid ultrasonic coupling material disposed between the ultrasonic detector and the first solid ultrasonic coupling material.

In some embodiments, wherein the detection device further comprises a sealing member, wherein both the first solid ultrasonic coupling material and the ultrasonic detector are disposed within an accommodation space of the sealing member; and the ultrasonic detector is capable of reciprocating within the sealing member along the second direction.

In some embodiments, wherein the detection device further comprises a support mesh disposed between the ultrasonic detector and the object.

Another aspect of the present disclosure may provide a medical imaging apparatus, which comprises: a detection device, wherein the detection device comprises: an X-ray emission source, configured to emit X-rays toward an object; an X-ray detector, movable along a first direction to acquire X-rays transmitted through the object; an ultrasonic detector, spaced apart from the X-ray detector and movable relative to the X-ray detector along a second direction to acquire ultrasonic signals from the object; and an imaging component, which displays at least one of an X-ray image associated with the X-rays and an ultrasonic image associated with the ultrasonic signals.

In some embodiments, wherein the medical imaging apparatus comprises a gantry, and the detection device is mounted on the gantry.

In some embodiments, wherein the gantry comprises a base and a support frame connected to one side of the base, the support frame extending outward relative to the base.

In some embodiments, wherein the detection device further comprises a compression unit arranged between the X-ray emission source and the X-ray detector.

In some embodiments, wherein the support frame has a sliding portion extending along a vertical direction, and the compression unit is slidably connected to the sliding portion.

In some embodiments, wherein the sliding portion is a sliding groove or a guide rail.

In some embodiments, wherein the medical imaging apparatus comprises an ultrasonic coupling medium, wherein at least a portion of the ultrasonic coupling medium is disposed between the ultrasonic detector and the object.

In some embodiments, wherein the ultrasonic coupling medium comprises a liquid coupling agent; wherein the medical imaging apparatus further comprises a container, wherein the liquid coupling agent is arranged within the container; and the ultrasonic detector is located within the container.

In some embodiments, wherein the ultrasonic coupling medium comprises a solid ultrasonic coupling material disposed between the ultrasonic detector and the object.

Another aspect of the present disclosure may provide a medical puncture system, which comprises an X-ray detector, movable along the first direction to acquire X-rays transmitted through an object; an ultrasonic detector, spaced apart from the X-ray detector and movable relative to the X-ray detector along the second direction; a puncture mechanism disposed above the X-ray detector and the ultrasonic detector.

In some embodiments, wherein the puncture mechanism comprises a puncture needle, and the puncture needle is drivable by the puncture mechanism to move toward the object along the first direction or the second direction.

### BRIEF DESCRIPTION OF THE DRAWINGS

In order to illustrate the embodiments of the present disclosure more clearly, the drawings used in the embodiments will be described briefly. Apparently, the following described drawings are merely for the embodiments of the present disclosure, and other drawings can be derived by those of ordinary skill in the art without any creative effort.

FIG. 1 is a schematic diagram of a detection device according to a first embodiment of the present application;

FIG. 2 is a schematic diagram of a detection device according to a second embodiment;

FIG. 3 is a schematic diagram of a detection device according to a third embodiment;

FIG. 4 is a schematic diagram of a detection device according to a fourth embodiment;

FIG. 5 illustrates the ultrasound detector in FIG. 4 moved to another position;

FIG. 6 shows a medical puncture system performing a puncture along the first direction;

FIG. 7 shows the medical puncture system in FIG. 6 performing a puncture along the second direction.

FIG. 8 is a schematic diagram of a medical imaging device according to an embodiment.

One or more embodiments of the present disclosure will be described in detail in the following figures and description. Other features, objects and advantages of this application will become more apparent from the description, drawings and claims.

### DETAILED DESCRIPTION

The present disclosure will now be described in detail with reference to the accompanying drawings and embodiments in order to make the objects, technical solutions, and advantages of the present disclosure more clear. It should be understood that the specific embodiments described herein are only for explaining the present disclosure, and not intended to limit the present disclosure.

In the description of this application, it should be understood that if terms such as “center,” “longitudinal,” “transverse,” “length,” “width,” “thickness,” “upper,” “lower,” “front,” “rear,” “left,” “right,” “vertical,” “horizontal,” “top,” “bottom,” “inner,” “outer,” “clockwise,” “counterclockwise,” “axial,” “radial,” or “circumferential” appear, these terms indicate orientations or positional relationships based on the orientation shown in the accompanying drawings. They are used solely to facilitate describing this application and simplifying the description, and do not indicate or imply that the referenced device or element must have a specific orientation or be constructed and operated in a specific orientation. Therefore, they should not be construed as limiting this application.

Furthermore, if terms such as “first,” “second,” etc., appear, these terms are for descriptive purposes only and should not be understood as indicating or implying relative importance or implicitly specifying the quantity of the indicated technical features. Thus, features defined as “first” or “second” may explicitly or implicitly include at least one such feature. In the description of this application, if the term “a plurality” appears, it means at least two, such as two, three, etc., unless explicitly and specifically defined otherwise.

In this application, unless otherwise expressly specified and defined, terms such as “installed,” “mounted,” “connected,” “coupled,” “fixed,” etc., should be interpreted broadly. For example, it may be a fixed connection, a detachable connection, or an integral connection; it may be a mechanical connection or an electrical connection; it may be a direct connection, an indirect connection through an intermediate medium, an internal connection between two elements, or an interaction relationship between two elements, unless otherwise expressly defined. For those of ordinary skill in the art, the specific meanings of the above terms in this application may be understood based on the specific context.

In this application, unless otherwise expressly specified and defined, if expressions such as a first feature being “on” or “under” a second feature appear, it may mean that the first and second features are in direct contact, or that the first and second features are in indirect contact through an intermediate medium. Moreover, expressions such as the first feature being “on,” “above,” or “over” the second feature may mean that the first feature is directly above, diagonally above, or merely that the horizontal level of the first feature is higher than that of the second feature. Expressions such as the first feature being “under,” “below,” or “beneath” the second feature may mean that the first feature is directly below, diagonally below, or merely that the horizontal level of the first feature is lower than that of the second feature.

It should be noted that if an element is referred to as being “fixed to” or “disposed on” another element, it may be directly on the other element or an intermediate element may be present. If an element is considered to be “connected” to another element, it may be directly connected to that element or an intermediate element may be present simultaneously. If present, terms such as “vertical,” “horizontal,” “upper,” “lower,” “left,” “right,” and similar expressions used in this application are for illustrative purposes only and do not represent the only possible implementation.

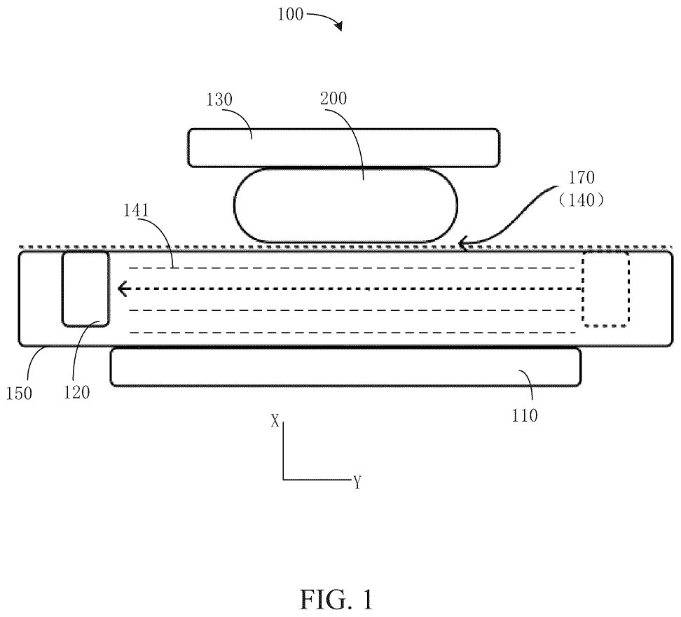

As shown in FIG. 1, a detection device 100 provided in an embodiment of this application includes a compression unit 130, an X-ray detector 110, and an ultrasound detector 120. The compression unit 130 is configured to compress (squeeze) and/or fix the object 200 to be examined. The X-ray detector 110 is positioned on one side of the compression unit 130 along a first direction X to acquire X-ray images of the object 200. The ultrasound detector 120 is spaced apart from the X-ray detector and is movable relative to the X-ray detector 110 along a second direction Y. In a practical application scenario, the first direction is vertical, and the second direction is horizontal, as indicated by the X and Y directions in FIG. 1. The compression unit 130 includes a compression paddle.

The detection device 100 enables both X-ray and ultrasound image acquisition. During X-ray examination of the object 200, the ultrasound detector 120 can move along the second direction Y outside the detection surface of the X-ray detector 110, ensuring that the ultrasound detector does not obstruct the X-ray detector from receiving X-rays, thereby allowing unimpeded X-ray examination. For ultrasound examination, the ultrasound detector 120 moves along the second direction Y to an appropriate position to emit ultrasonic waves for imaging. Throughout this process, X-ray and ultrasound image acquisitions are mutually non-interfering. This allows for the identification of pathological details (e.g., microcalcifications) via X-ray imaging while visualizing the complete structural imaging of lesions via ultrasound, enhancing the flexibility of the detection device 100. Additionally, the time required for patients to complete both examinations is significantly reduced, improving overall efficiency.

In some embodiments, the X-ray detector 110 may convert X-ray energy into recordable electrical signals. It receives X-ray irradiation and generates electrical signals proportional to the radiation intensity. The signal strength received by the X-ray detector 110 depends on the density of tissues within the cross-section of the object 200. For example, high-density tissues (e.g., bones) absorb more X-rays, resulting in weaker signals detected by the X-ray detector 110, whereas low-density tissues (e.g., fat) absorb fewer X-rays, producing stronger signals. Thus, the signal strength reflects the state of human tissues. The X-ray detector 110 may be a flat-panel detector, and its detection surface corresponds to the functional area within the panel where photoelectric conversion occurs. Structurally, the detection surface excludes the panel's frame and includes only the region with photoelectric conversion components.

In some embodiments, the ultrasound detector 120 scans the human body with ultrasonic beams and processes reflected signals to generate images of internal organs. During ultrasound examination, air between the ultrasound probe and the patient's skin may impede ultrasonic wave transmission. As shown in FIG. 1, the detection device 100 may further include an ultrasound coupling medium 140 positioned between the ultrasound detector 120 and the object 200 to enhance imaging quality. The coupling medium 140 bridges the ultrasound probe and the patient's skin, improving detection efficacy. The coupling medium 140 may include low-mobility agents such as water-based gels, gel-based couplants, or oil-based couplants.

In some embodiments, the ultrasound detector 120 is connected to a driving mechanism (not shown) that moves the ultrasound detector 120 along the second direction Y to either avoid interference with the X-ray detector 110 or position it optimally for examination. The driving mechanism may include robotic arms or linear actuators to adjust the position of the ultrasound detector 120.

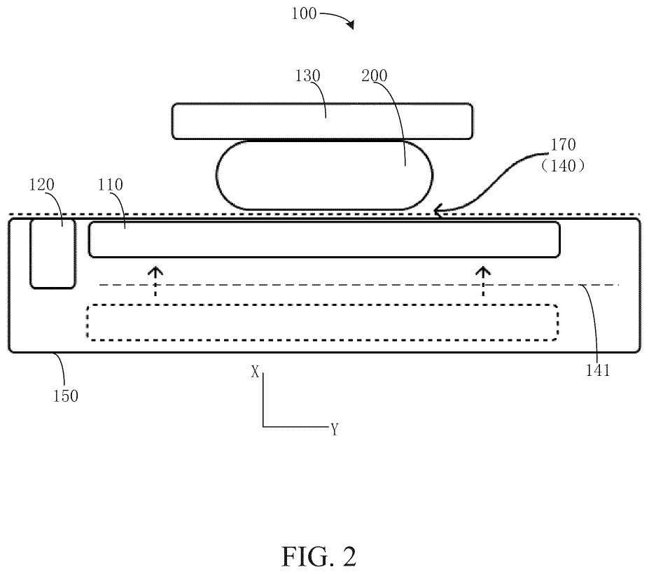

As shown in FIG. 2, in one embodiment, the X-ray detector 110 is movable relative to the compression unit 130 along the first direction X to adjust the distance between the X-ray detector 110 and the object 200. By positioning the X-ray detector 110 closer to the object 200, X-ray attenuation is reduced, thereby improving X-ray image quality. A driving mechanism is connected to the X-ray detector 110 to enable its movement along the first direction X.

As shown in FIG. 1 or FIG. 2, in one embodiment, the ultrasound coupling medium 140 includes a liquid couplant 141. The detection device 100 further includes a container 150, with both the liquid couplant 141 and the ultrasound detector 120 positioned within the container.

The container 150 forms a sealed space to hold the liquid couplant 141, ensuring air exclusion and maintaining ultrasound image quality. The container 150 may have a rectangular cross-section (as in this embodiment) and can be made of alloy or polyester materials. The liquid couplant 141 may include mineral oil.

As shown in FIG. 2, in another embodiment, both the ultrasound detector 120 and the X-ray detector 110 are housed within the container 150. The X-ray detector 110 moves along the first direction X to minimize X-ray attenuation and enhance image quality. The X-ray detector 110 and the ultrasound detector 120 may be connected to the inner walls of the container 150, with their positional adjustments achieved through relative motion. The driving mechanisms for their movement may also be integrated within the container 150.

As shown in FIG. 2, in one embodiment, the detection surface of the X-ray detector 110 faces the inner side of the container 150 (e.g., the top panel). The ultrasound detector 120 is movably positioned within the container 150 and can be pressed against the inner surface. X-rays pass through the inner surface of the container 150 to reach the X-ray detector 110, while the ultrasound detector 120 moves within the container 150 to align with the inner surface for imaging. When the object 200 (e.g., breast tissue) is placed against the outer wall of the container 150, the ultrasound detector 120 can perform examinations through the inner surface.

As shown in FIG. 2, in some embodiments, the inner surface of the container 150 and the X-ray detector 110 may be spaced apart, i.e., a space is reserved between the inner surface of the container 150 and the X-ray detector 110. The vertical distance between the inner surface of the container 150 and the X-ray detector 110 can be adjusted as needed. The ultrasonic detector 120 can move within a horizontal plane between the inner surface of the container 150 and the X-ray detector 110. When X-ray detection of the object 200 is required, the ultrasonic detector 120 can be moved within the horizontal plane between the inner surface of the container 150 and the X-ray detector 110 to position the ultrasonic detector 120 away from the object 200 and the X-ray detector 110. This ensures that the ultrasonic detector 120 does not obstruct the reception of X-rays by the X-ray detector 110. At this time, the ultrasonic detector 120 does not interfere with the operation of the X-ray detector 110. When ultrasonic detection of the object under test 200 is required, the ultrasonic detector 120 can be moved to a position between the X-ray detector 110 and the inner surface of the container 150. Since the X-ray detector 110 is inactive in this scenario, the ultrasonic detector 120 can move above the X-ray detector 110 and emit ultrasonic waves to inspect the object 200.

As shown in FIG. 2, in one embodiment, the detection surface of the ultrasonic detector 120 may be parallel to the inner surface of the container 150. The movement path of the ultrasonic detector 120 within the horizontal plane between the X-ray detector 110 and the inner surface of the container 150 is not limited, provided that the ultrasonic detector 120 and the X-ray detector 110 do not interfere with each other during their respective operations. A slide track (not shown) may be disposed on the plane between the X-ray detector 110 and the inner surface of the container 150, allowing the ultrasonic detector 120 to slide along the slide track.

As shown in FIG. 2, in some embodiments, the X-ray detector 110 is movably arranged along a vertical direction within the accommodation space. Specifically, when the inner surface of the container 150 is horizontally disposed, the X-ray detector 110 can move vertically toward or away from the inner surface of the container 150. During X-ray detection of the object 200, the X-ray detector 110 can be vertically moved closer to the inner surface of the container 150 to improve the accuracy of X-ray reception. When the X-ray detector 110 is inactive, it can be moved away from the inner surface of the container 150, leaving a larger space between them. In this state, the ultrasonic detector 120 can be moved horizontally between the X-ray detector 110 and the inner surface of the container 150, enabling ultrasonic detection of the object 200 placed on the inner surface of the container 150. It is noted that a vertically oriented slide rail (not shown) may be disposed within the accommodation space, with the X-ray detector 110 slidably mounted on the rail. The slide rail may include multiple spaced locking structures to secure the position of the X-ray detector 110, thereby defining its vertical distance relative to the inner surface of the container 150.

In one embodiment, the detection device further includes a first horizontal slide rail (not shown) disposed between the inner surface of the container and the X-ray detector. The ultrasonic detector is slidably mounted on the first slide rail. It is understood that the ultrasonic detector may be connected to a driving mechanism, enabling motorized movement of the ultrasonic detector.

In one embodiment, the detection device further includes a second slide rail (not shown) vertically arranged within the accommodation space, with the X-ray detector slidably mounted on the second slide rail. The X-ray detector can slide vertically along the second slide rail to adjust its vertical distance from the object, thereby enhancing the accuracy of X-ray reception.

As shown in FIG. 2, in one embodiment, the ultrasonic detector 120 is in close contact with the inner surface of the container 150, thereby positioning the ultrasonic detector 120 closer to the object 200 for improved detection performance. Additionally, since the ultrasonic detector 120 is attached to the inner surface of the container 150, the inner surface defines the horizontal plane for the movement of the ultrasonic detector 120, facilitating guidance and control of its motion. This configuration also enhances the compactness of the detection device 100, reducing the space occupied by the mechanism.

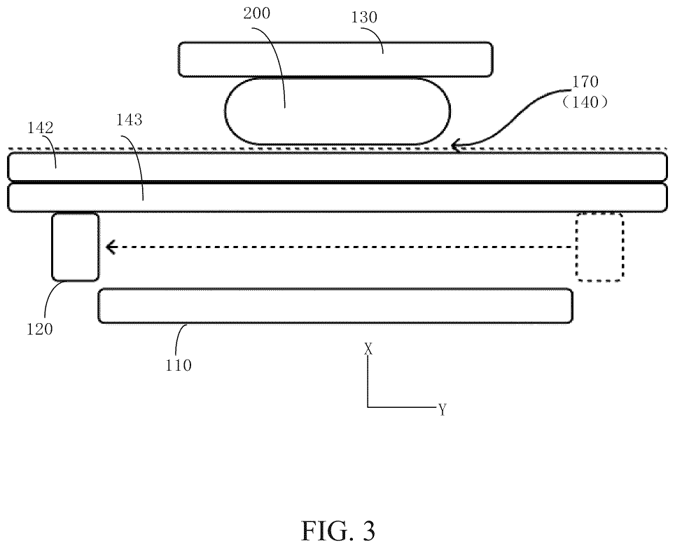

As shown in FIG. 3, in one embodiment, the container 150 described earlier may be omitted. The ultrasonic coupling medium 140 includes a first solid ultrasonic coupling material 142 disposed between the ultrasonic detector 120 and the object 200. The first solid ultrasonic coupling material 142 connects the ultrasonic probe to the patient's skin surface, enhancing ultrasonic detection effectiveness and producing high-quality ultrasound images. The first solid ultrasonic coupling material 142 supports the compressed object and may be made of a rigid material with moderate stiffness. For example, the first solid ultrasonic coupling material 142 could be a highly crystalline transparent plastic material, such as polymethyl pentene (TPX), which optimizes ultrasonic wave propagation to improve image quality and diagnostic accuracy.

As shown in FIG. 3, in one embodiment, the ultrasonic coupling medium 140 further includes a second solid ultrasonic coupling material 143 disposed between the ultrasonic detector 120 and the first solid ultrasonic coupling material 142. The second solid ultrasonic coupling material 143 is made of a flexible material and adheres to the surface of the first solid ultrasonic coupling material 142. This mitigates potential coupling inefficiencies between the rigid first solid ultrasonic coupling material 142 and the ultrasonic detector 120 caused by poor surface wettability. The combination of the first and second solid ultrasonic coupling materials 142, 143 further enhances ultrasound image quality. The second solid ultrasonic coupling material 143 may act as an acoustic coupling pad, serving as an acoustic interface between the ultrasonic probe and the patient's skin. It eliminates air gaps between the probe and skin, ensuring efficient transmission of ultrasonic waves into the patient's body and reducing signal attenuation at air interfaces, thereby improving image clarity. The acoustic coupling pad also extends the near-field distance of the ultrasonic probe, enabling lesion tissues to reside within the focused beam area for enhanced detection capability.

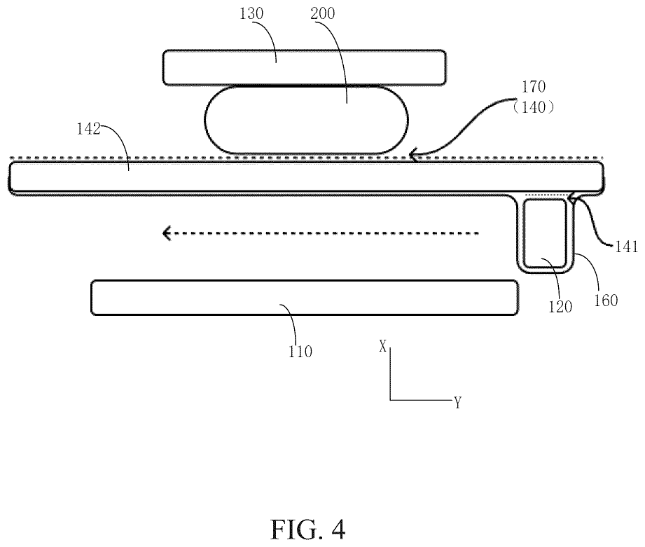

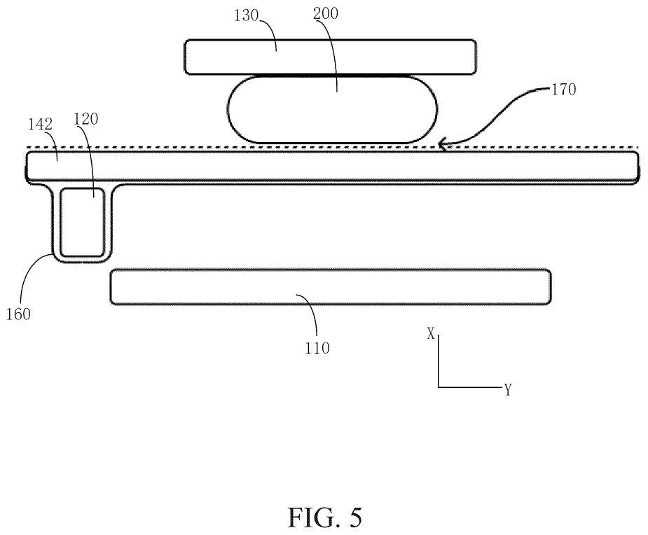

As shown in FIGS. 4 and 5, in one embodiment, the detection device 100 includes a sealing member 160 filled with a liquid coupling agent 141. Both the first solid ultrasonic coupling material 142 and the ultrasonic detector 120 are housed within the sealed cavity of the sealing member 160. The ultrasonic detector 120 is capable of reciprocating along a second direction within the sealing member 160. The sealing member 160 may adopt a sealed synchronous belt structure, where movement of the ultrasonic detector 120 along the second direction causes synchronized motion of the sealing member 160. Within the cavity of the sealing member 160, the first solid ultrasonic coupling material 142 and the ultrasonic detector 120 are surrounded by the liquid coupling agent 141, such as a water-based gel, oil-based, or other coupling agents, to eliminate air gaps between them and enhance coupling efficiency. The ultrasonic detector 120 and the sealing member 160 may synchronize motion through an external linear actuator, such as a linear motor or pneumatic cylinder. For example, the ultrasonic detector 120 may be connected to the actuator to drive both itself and the sealing member 160, or conversely, the sealing member 160 may be driven to move the ultrasonic detector 120. This allows the ultrasonic detector 120 to move outside the detection plane of the X-ray detector 110 along the second direction, avoiding interference with X-ray detection. Alternatively, the ultrasonic detector 120 can be positioned optimally for emitting ultrasonic waves toward the object under test 200. The driving method may be customized based on practical needs. The use of solid ultrasonic coupling materials reduces X-ray attenuation, improves X-ray image quality, and ensures a more compact layout of the detection device 100.

As shown in FIG. 5, in one embodiment, the detection device 100 further includes a support mesh 170 disposed between the ultrasonic detector 120 and the object under test 200. The support mesh 170 is coated with a portion of the ultrasonic coupling medium 140, such as hydrogel. Taking breast imaging as an example, during mammography, compression of the breast is required for imaging. The ultrasonic coupling medium 140 may render the breast surface slippery, complicating positioning during compression. The addition of the support mesh 170 not only maintains air isolation but also increases friction between the object 200 and the ultrasonic detector 120, facilitating stable positioning. The support mesh 170 may specifically be a gauze-like mesh structure.

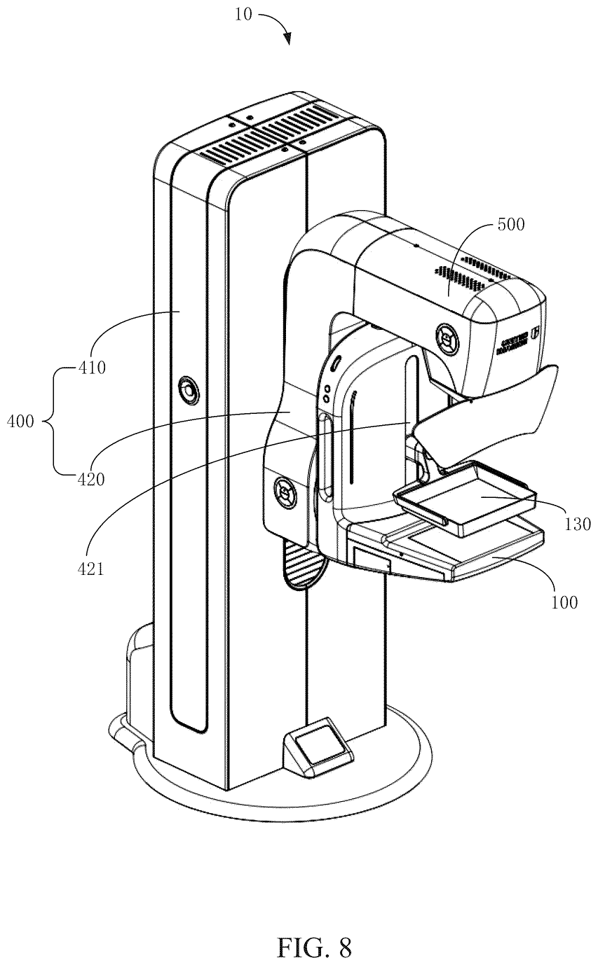

As shown in FIG. 8, further, an embodiment of the present application provides a medical imaging device 10, comprising an imaging component and the aforementioned detection device 100. Understandably, it also includes an X-ray emission source 500, such as an X-ray tube. The object 200 may be placed between the X-ray emission source 500 and the X-ray detector 110. X-rays emitted by the X-ray emission source 500 pass through the object 200 and are received by the X-ray detector 110. Combined with the imaging component, X-ray images and ultrasound images can be displayed. When in use, the medical imaging device 10 can simultaneously acquire X-ray images and ultrasound images without mutual interference, thereby enhancing operational flexibility. Additionally, the time required for patients to complete both X-ray and ultrasound examinations is significantly reduced, improving examination efficiency. The imaging component may be a display screen, allowing operators to observe detection images through the screen. In a practical application scenario, the medical imaging device may be a mammography machine.

As shown in FIG. 8, in one embodiment, the medical imaging device 10 includes a frame 400, with the detection device 100 mounted on the frame 400. The frame 400 supports the detection device 100, ensuring stability even during movement to prevent image blurring and enabling high-quality detection images.

Specifically, as shown in FIG. 8, the frame 400 includes a base 410 and a support arm 420 connected to one side of the base 410. The support arm 420 extends from the base 410, and the detection device 100 is mounted on the support arm 420. The X-ray emission source 500 is positioned at the upper end of the support arm 420, while the detection device 100 is located at the lower end. X-rays emitted by the X-ray emission source 500 pass through the object and are received by the X-ray detector within the detection device 100.

As shown in FIG. 8, further, the support arm 420 is provided with a sliding portion 421 extending vertically. The compression unit 130 is slidably connected to the sliding portion 421. By moving the compression unit 130 vertically along the sliding portion 421, its position can be adjusted to compress the object, ensuring uniform compression of the target tissue. This reduces tissue thickness, improves image quality, and minimizes radiation dosage. The sliding portion 421 may specifically be a groove or rail structure that accommodates the movement of the compression unit 130. The compression unit may include a compression paddle, which is integrated with an ultrasound transducer.

In further embodiments, shielding materials may be incorporated within the frame to reduce the impact of scattered X-rays on the surrounding environment, safeguarding the health of operators and the object.

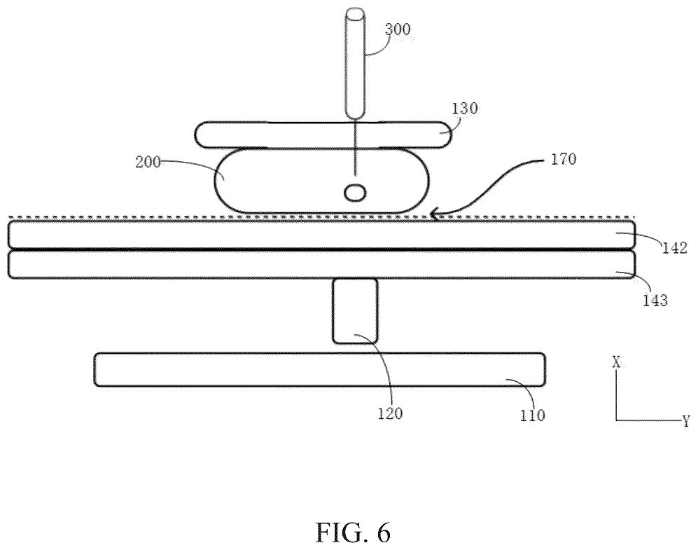

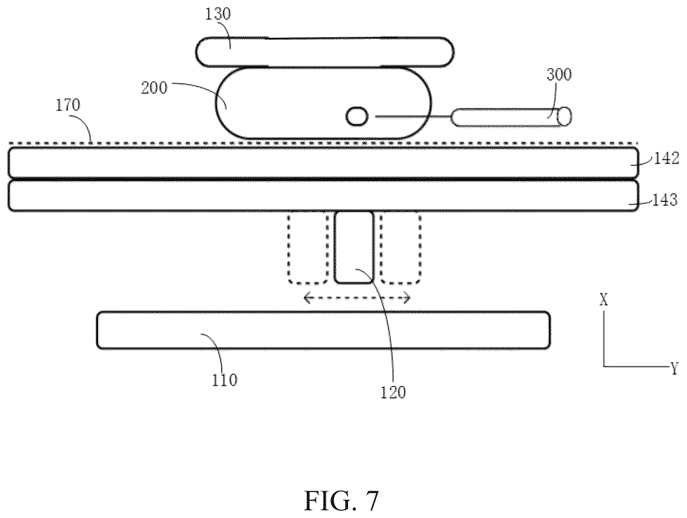

As shown in FIGS. 6 and 7, an embodiment of the present application further provides a medical puncture system, comprising a puncture mechanism and the aforementioned medical imaging device. In a specific embodiment, the medical puncture system may be a breast biopsy system, where the puncture mechanism includes a needle-holding instrument for gripping a puncture needle 300. The puncture direction of the puncture needle 300 can be selected based on practical needs. For example, in the embodiment shown in FIG. 6, the puncture needle 300 advances vertically, while in the embodiment shown in FIG. 7, the puncture needle 300 advances horizontally.

The described medical puncture system integrates the ultrasonic detector and X-ray detector. The X-ray detector enables localization of the lesion site, i.e., the three-dimensional coordinates of the pathological tissue, thereby determining the puncture depth of the needle. The needle-holding instrument can guide the puncture needle directly to the target tissue. Simultaneously, the entire puncture process is visualized via ultrasound imaging, allowing real-time observation of both the needle's position and the lesion location. This eliminates the need for repeated manual adjustments of the ultrasonic detector by physicians to track the needle and tissue positions, ensuring precise intracavitary puncture operations with enhanced accuracy and efficiency. Specifically, spatial alignment between the X-ray and ultrasonic detectors is achieved through algorithmic coordination of image coordinates. When viewing X-ray images, the system automatically synchronizes ultrasound images, enabling lesion points identified on X-ray images to be tracked and displayed on the ultrasound interface, further improving puncture precision.

The aforementioned detection device enables both X-ray image acquisition and ultrasound image acquisition. During X-ray examination of the object to be examined, the ultrasound detector can move along the second direction outside the detection surface of the X-ray detector, ensuring that the ultrasound detector does not obstruct the X-ray detector from receiving X-rays, thereby allowing unimpeded X-ray examination. During ultrasound examination of the object, the ultrasound detector can move along the second direction to an appropriate position to emit ultrasonic waves for the examination. Throughout this process, X-ray image acquisition and ultrasound image acquisition operate independently without mutual interference. This allows for the identification of pathological details such as microcalcifications through X-ray imaging while visualizing the complete structural imaging of pathological tissues via ultrasound, thereby enhancing the operational flexibility of the detection device. Additionally, the time required for patients to complete both X-ray and ultrasound examinations is significantly reduced, improving examination efficiency.

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure. Therefore, the patent protection of the present disclosure shall be defined by the appended claims.

What is claimed is:

1. A detection device, comprising:
a compression unit, configured to compress an object;
an X-ray detector, located on one side of the compression unit along a first direction, the X-ray device adapted for acquiring an X-ray image of the object;
an ultrasonic detector, spaced apart from the X-ray detector and capable of moving relative to the X-ray detector along a second direction, the ultrasonic detector adapted for acquiring an ultrasonic image of the object.

2. The detection device as claimed in claim 1, wherein the X-ray detector is movable relative to the compression unit along the first direction; wherein ultrasonic detector and the X-ray detector are positioned along the same side of the compression assembly relative to the first direction.

3. The detection device as claimed in claim 1, further comprising an ultrasonic coupling medium, wherein at least a portion of the ultrasonic coupling medium is disposed between the ultrasonic detector and the object.

4. The detection device as claimed in claim 3, wherein the ultrasonic coupling medium comprises a liquid coupling agent; wherein the detection device further comprises a container, wherein the liquid coupling agent is arranged within the container; and wherein the ultrasonic detector is located within the container.

5. The detection device as claimed in claim 4, wherein the X-ray detector is located within the container.

6. The detection device as claimed in claim 3, wherein the ultrasonic coupling medium comprises a first solid ultrasonic coupling material disposed between the ultrasonic detector and the object.

7. The detection device as claimed in claim 6, wherein the ultrasonic coupling medium further comprises a second solid ultrasonic coupling material disposed between the ultrasonic detector and the first solid ultrasonic coupling material.

8. The detection device as claimed in claim 6, further comprising a sealing member, wherein both the first solid ultrasonic coupling material and the ultrasonic detector are disposed within an accommodation space of the sealing member; and the ultrasonic detector is capable of reciprocating within the sealing member along the second direction.

9. The detection device as claimed in claim 1, further comprising a support mesh disposed between the ultrasonic detector and the object.

10. A medical imaging apparatus, comprises:
a detection device, wherein the detection device comprises:
an X-ray emission source, configured to emit X-rays toward an object;
an X-ray detector, movable along a first direction to acquire X-rays transmitted through the object;
an ultrasonic detector, spaced apart from the X-ray detector and movable relative to the X-ray detector along a second direction to acquire ultrasonic signals from the object; and
an imaging component, displaying at least one of an X-ray image associated with the X-rays and an ultrasonic image associated with the ultrasonic signals.

11. The medical imaging apparatus as claimed in claim 10, wherein the medical imaging apparatus comprises a gantry, and the detection device is mounted on the gantry.

12. The medical imaging apparatus as claimed in claim 11, wherein the gantry comprises a base and a support frame connected to one side of the base, the support frame extending outward relative to the base.

13. The medical imaging apparatus as claimed in claim 12, wherein the detection device further comprises a compression unit arranged between the X-ray emission source and the X-ray detector.

14. The medical imaging apparatus as claimed in claim 13, wherein the support frame has a sliding portion extending along a vertical direction, and the compression unit is slidably connected to the sliding portion.

15. The medical imaging apparatus as claimed in claim 14, wherein the sliding portion is a sliding groove or a guide rail.

16. The medical imaging apparatus as claimed in claim 10, further comprising an ultrasonic coupling medium, wherein at least a portion of the ultrasonic coupling medium is disposed between the ultrasonic detector and the object.

17. The medical imaging apparatus as claimed in claim 16, wherein the ultrasonic coupling medium comprises a liquid coupling agent; wherein the medical imaging apparatus further comprises a container, wherein the liquid coupling agent is arranged within the container; and the ultrasonic detector is located within the container.

18. The medical imaging apparatus as claimed in claim 17, wherein the ultrasonic coupling medium comprises a solid ultrasonic coupling material disposed between the ultrasonic detector and the object.

19. A medical puncture system, comprising:
an X-ray detector, movable along the first direction to acquire X-rays transmitted through an object;
an ultrasonic detector, spaced apart from the X-ray detector and movable relative to the X-ray detector along the second direction;
a puncture mechanism disposed above the X-ray detector and the ultrasonic detector.

20. The medical puncture system as claimed in claim 19, wherein the puncture mechanism comprises a puncture needle, and the puncture needle is drivable by the puncture mechanism to move toward the object along the first direction or the second direction.
